(19) United States
(12) Patent Application Publication
(10) Pub. No.: US 2023/0408516 A1
(43) Pub. Date: Dec. 21, 2023

(54) METHOD OF MAKING A PORTABLE MIP-BASED ELECTROCHEMICAL SENSOR FOR THE DETECTION OF THE SARS-COV-2 ANTIGEN

(71) Applicant: TALLINN UNIVERSITY OF TECHNOLOGY, Tallinn (EE)

(72) Inventors: Vitali SÕRITSKI, Tallinn (EE); Jekaterina REUT, Tallinn (EE); Roman BOROZNJAK, Tallinn (EE); Anna KIDAKOVA, Tallinn (EE); Andres ÖPIK, Tallinn (EE); Abdul RAZIQ, Tallinn (EE); Kaspar RATNIK, Tallinn (EE); Paul NAABER, Tallinn (EE)

(21) Appl. No.: 18/036,063

(22) PCT Filed: Nov. 10, 2021
(86) PCT No.: PCT/IB2021/060399

§ 371 Date: May 9, 2023

Related U.S. Application Data
(60) Provisional application No. 63/112,109, filed on Nov. 10, 2020.

(51) Int. Cl. G01N 33/569; G01N 33/543

(52) U.S. Cl. CPC G01N 33/56983; G01N 33/5438; G01N 2600/00; G01N 2469/10; G01N 2333/165

(57) ABSTRACT

The current COVID-19 pandemic caused by SARS-CoV-2 coronavirus is expanding around the globe. Hence, accurate and cheap portable sensors are crucially important for the clinical diagnosis of COVID-19. Molecularly imprinted polymers (MIPs) as robust synthetic molecular recognition materials with antibody-like ability to bind and discriminate between molecules are provided here as selective elements in such sensors. Provided are detection assemblies comprising electrochemical sensors having ncovNP-MIP film endowed selectivity against SARS-CoV-2 nucleoprotein (ncovNP) and/or ncovS1-MIP film endowed selectivity against SARS-CoV-2 spike 1 (S1). The ncovNP- or ncovS1-MIP are synthesized electrochemically on portable gold thin-film electrodes system via chronocoulometry or cyclic voltammetry. The sensors show excellent detection capabilities, and high discrimination of interfering proteins.

### FIELD OF THE INVENTION

This invention relates generally to detection of antigens, more specifically to detection of antigens with molecularly imprinted polymers (MIP) and even more specifically detection of SARS-CoV-2 antigens with MW-based sensors.

### BACKGROUND OF THE INVENTION

The ongoing outbreak of COVID-19 experienced around the globe was discovered in December 2019 in Wuhan, China [1]. WHO officially declared it pandemic on 12 Mar. 2020 [2]. The primary symptoms of COVID-19 infection are fever, coughing, shortness in breathing, etc. However, in some cases, patients may be asymptomatic, with no coughing, and fever or have mild symptoms. These asymptomatic patients have the greater potential to spread the disease quickly to the other peoples. Therefore, in order to trace and diagnose COVID-19 patients, rapid and accurate screening of COVID-19 carriers is of crucial importance for the prevention of spreading of the virus at early stages [3].

Severe acute respiratory syndrome coronavirus 2 (SARS-CoV-2) belongs to the enveloped, positive-stranded RNA virus family. SARS-CoV-2 has four major structural proteins: spike, membrane, envelope, and nucleocapsid [4,5]. The SARS-CoV-2 nucleocapsid protein (ncovNP) is responsible for packaging and protecting coronavirus genomic RNA [6-8]. The high abundance and immunogeni city of ncovNP make it a suitable antigen for the development of COVID-19 diagnostic tests [5]. Thus, for example, the high diagnostics value of serum ncovNP in the early stage of infection was confirmed by ELISA double antibody sandwich assay [9]. The SARS-CoV-2 spike protein subunit S1 (ncovS1) is the major surface antigen of SARS-CoV-2 being a component for the trimeric spike protein complex [35]. S1 is the major target antigen for vaccine development against SARS-CoV-2 [36]

Today, the reverse transcription polymerase chain reaction (RT-PCR) is one of the most accurate laboratory methods for detecting SARS-CoV-2 from the samples like nasopharyngeal swab of patients and is used for routine diagnosis of COVID-19 in many laboratories worldwide [10]. However, RT-PCR tests require expensive instrumentation and skilled personnel, have a long turnaround time and complex protocols. Moreover, these tests may be prone to the false-negative results. Apart from that, ELISA methods have also been developed for SARS-CoV-2 antibodies testing in serum [11]. Although the serological tests are cheaper and have shorter analysis time as compared to molecular tests, they are not suitable for the diagnosis at early-stage of infection, since the detectable level of antibodies is produced at 10-14 days after the onset of the symptoms and can therefore be used mainly for serological screening and epidemiological studies [12]. In addition, SARS CoV-2 detection from wastewater or other environmental samples enables to track the community infection dynamics, but these samples are very prone to PCR inhibition [37]. Therefore, there is a crucial demand in cheaper, portable biosensing devices to facilitate rapid testing for COVID-19 and the presence of SARS CoV-2 antigens in various types of samples. Recently, different testing technologies for rapid detection of SARS-CoV-2 specific antigens (a viral protein) have been developed and some of them are already commercially available [13]. Porte, L. et al reported on the development of rapid SARS-CoV-2 antigen test based on fluorescence immunochromatographic assay [14]. Seo et al reported a field-effect transistor (FET)-based biosensing device for detecting SARS-CoV-2 spike protein in clinical samples [15]. An FDA approved commercially available test Sofia SARS antigen fluorescent immunoassay is based on advanced immunofluorescence lateral flow technology in a sandwich design and allows qualitative detection of nucleocapsid protein from SARS-CoV-2 [16].

However, most of these diagnostic tools rely on biological recognition elements, i.e., diagnostic antibodies that ensure the selectivity of the device towards the target but reduce sensor shelf life and enhance the cost. Thus, there is a need for alternative diagnostic tools that would not rely on biological recognition elements.

Molecularly imprinted polymers (MIPs)—materials have been experimented to some degree as synthetic receptors. Molecular imprinting can be defined as a process of template-induced formation of specific molecular recognition sites in a polymer material. In this process, a mixture of functional monomers is polymerized around a chosen target acting as a template. Subsequent removal of the templates from the formed polymer leaves behind binding sites that are complementary to the target molecule in size, shape arrangement of functional groups and capable of selectively recognizing these molecules. The main benefits of MIPs as synthetic receptors are excellent chemical and thermal stability coupled with their reproducible and cost-effective fabrication [17,18]. Therefore, MIPs integrated with different sensing platforms have been studied in recent decades for detection of various disease biomarkers such as e.g. epithelial ovarian cancer antigen [19], cancer biomarker-prostate specific antigen [20], cancer tumor marker CA 15-3 [21], myoglobin [22], and cardiac troponin T [23]. Recently development of MIP-based sensors for the detection of neurotrophic factor proteins such as BDNF and CDNF as the potential biomarkers of the neurodegenerative diseases has been reported [24,25]. Furthermore, MIP-based sensors have also been investigated for the detection of certain viral proteins: Lu et al. developed glycoprotein selective MIP on quartz crystal microbalance (QCM) for diagnosis of human immunodeficiency virus type 1 [26]. Tai et al. fabricated NSlprotein selective MIP on QCM for detection of dengue virus by using epitope-imprinting approach coupled by chemical or photopolymerization [27]. The current COVID-19 pandemic caused by SARS-CoV-2 coronavirus is expanding around the globe. Hence, accurate and affordable portable sensors are crucially important for the clinical diagnosis of COVID-19.

The Molecularly imprinted polymers (MIPs) as robust synthetic molecular recognition materials with antibody-like ability to bind and discriminate between molecules can perfectly serve for building selective elements in such sensors. However, to date, according to our knowledge, the detection of ncovNP or ncovS1 by a MIP-based electrochemical sensor has never been reported in the literature. Furthermore, the detection sensors provided here use protein-surface imprinting approach coupled with more controllable electropolymerization method to deposit polymer directly on the sensor transducer and give better control over the thickness of the polymer layer

### SUMMARY OF THE INVENTION

With the above in mind, this disclosure now provides surprising and novel solutions over the above-mentioned limitations, and over the existing prior art, and particularly, provides solutions for developing accurate portable detection assemblies for detecting SARS-CoV-2 positive samples and methods for making the detection assemblies as well as method of detecting positive samples.

Accordingly, it is an object of this invention to provide a detection assembly for detecting at least one SARS-CoV-2 antigen from a sample, the assembly comprising a portable electrochemical sensor integrated with a SARS-CoV-2 antigen -molecular imprinted polymer (MIP), wherein the SARS-CoV-2 antigen-MW is configured to act as a synthetic recognition element selectively detecting and binding the at least one SARS-CoV-2 antigen, and a reading device capable of measuring presence or absence of the antigen.

In certain embodiments of the detection assembly the at least one SARS-CoV-2 antigen is SARS CoV-2 nucleoprotein ncovNP. In certain embodiments the SARS-CoV-2 antigen is SARS CoV-2 S1-subunit protein ncovS1.

The sample from which the detection is conducted may be from a human source, such as a swabbing sample from mucous membranes, or blood, urine, or saliva sample. The sample from which the detection is conducted may be from an environmental source, such as sewage water, or air to liquid sedimentation sampler. The sample from which the detection is conducted may be from a laboratory source, such as a recombinant protein expression or purification sample.

The detection assembly according to certain embodiments has a detection limit in a range of 15 to 70 fM, more preferably 15 to 50 fM and most preferably 15 fM-30 fM.

The detection assembly according to certain embodiments has a quantification limit in the range of 50 to 220 fM, more preferably 50 to 80 fM, and most preferably about 50-51 fM.

According to certain embodiments the detection assembly has a shelf life more than 5 weeks, preferably more than 7 weeks, and most preferably at least 9 weeks.

According to certain embodiments the detection assembly is configured to discriminate at least E2 (E2 envelope protein of Hepatitis C virus), HCV (Hepatitis C virus antigens), BSA (bovine serum albumin), HSA (human serum albumin), IgG (immunoglobulin G) and CD48 (Cluster of Differentiation 48) proteins, whereby the assembly is reliable and is expected to have a low false positive counts.

According to certain embodiments of the detection assembly, a polymeric layer coating of the electrochemical sensor is formed from electropolymerizable monomers selected from the group consisting of mPD, 3-aminophenylboronic acid (APBA), dopamine and EDOT; most preferably mPD or APBA, and the polymeric layer coating is deposited by 1-10 mC/cm²; more preferably 1-7 mC/cm², and most preferably by 2-7 mC/cm²

According to certain embodiments the assembly comprises a portable potentiostat reader.

According to certain embodiments the potentiostat reader may be connected to a portable personal computer, such as cell phone.

It is an object of this invention to provide a method to detect presence or absence or the quantitative concentration of at least one SARS-CoV-2 antigen in a sample, wherein the method comprises: providing a sensor comprising SARS-Cov-2 antigen molecular imprinted polymer (MIP) integrated with a sensing electrode, such as a thin film electrode (TFE); bringing the sensor in contact with the sample; and detecting the presence or absence of the antigen by differential pulse voltammetry (DPV) or square wave voltammetry (SWV), wherein presence of the antigen is recorded when limit of detection (LOD) of the sensor is exceeded.

According to certain embodiments the method includes contacting the sensor with the sample by incubating the sensor in a lysis buffer containing detergent or other even simpler buffers without the detergent (such as PBS—phosphate buffered saline) which comprises the sample.

In certain embodiments the incubation time is less than an hour, more preferably less than 30 minutes, even more preferably between 20 minutes to 30 minutes, and most preferably 15-20 minutes.

According to certain embodiments of the method the antigen is SARS CoV-2 nucleoprotein ncovNP or/and SARS CoV-2 S1-subunit protein ncovS 1.

According to certain aspects, the method may include obtaining the sample from an environmental source, such as sewage water, or air to liquid sedimentation sampler. According to some other aspects the method may include obtaining the sample from a human source, such as a swabbing sample from mucous membranes, or blood, urine or saliva sample. According to certain aspects the method may include obtaining the sample from a laboratory source, such as a recombinant protein expression or purification sample.

It is a further object of the invention to provide a method of making an electrochemical sensor for detection SARS-CoV-2 antigen from samples, wherein the antigen is ncovNP or ncovS1, the method comprising the steps of: forming a cleavable linking layer on a metallic surface e.g. gold of a sensing electrode deposited on an insulating support e.g. glass; immobilizing ncovNP and/or ncovS1 molecules on the cleavable linking layer on the electrode surface; polymerizing a polymer on the antigen-immobilized electrode surface thereby forming a polymeric layer coating on the electrode surface with entrapped antigen molecules; and cleaving off the cleavable linking layer thereby removing the antigen molecules from the polymeric layer and obtaining antigen sensor configured to capture antigens similar to those removed by cleaving off the linking layer.

According to certain aspects the polymeric layer coating is formed from electropolymerizable monomers selected from the group consisting of m-phenylenediamine (mPD), 3-aminophenylboronicacid (APBA), dopamine and 3,4-ethylenedioxythiophene (EDOT), most preferably mPD or APBA. According to certain aspects the polymer film (polymer layer coating) is deposited by charge density of 1-10 mC/cm² more preferably 1-7 mC/cm². Most preferably the film is deposited by charge density of 2-7 mC/cm².

A portable electrochemical sensor for use in detection assembly for detection of SARS-CoV-2 antigens, ncovNP and/or ncovS1 is provided here. The sensors utilize a synthetic receptor—a polymer film molecularly imprinted with ncovNP (ncovNP-MIP) and/or ncovS1 (ncovS1-MIP). The sensors demonstrate detection of SARS-CoV-2 antigen ncovNP and ncovS1, respectively, at fM level. The sensor performance was validated by using nasopharyngeal swab samples and laboratory recombinant protein samples. It can be assumed by the principle, that the antigens are expected to be detected by the sensor from other samples than what are exemplary wise shown here.

The current COVID-19 pandemic caused by SARS-CoV-2 coronavirus is expanding around the globe. Hence, accurate, economic portable sensors with long shelf life are crucially important for various purposes: from monitoring of the viral spread and load in wastewater systems, for clinical diagnosis of COVID-19, and up to the use in research, development or production environment to detect SARS-CoV-2 antigens, for example during the vaccine development. Molecularly imprinted polymers (MIPs) as robust synthetic molecular recognition materials with antibody-like ability to bind and discriminate between molecules can perfectly serve for building selective elements in such sensors.

Provided here is an electrochemical sensor that comprises a ncovNP-MIP and/or ncovS1-MIP film endowed selectively against antigens SARS-CoV-2 nucleoprotein (ncovNP) or spike protein (ncovS1), respectively, and is configured to signal the presence of the antigen in COVID-19 positive samples.

The ncovNP-MIP and ncovS1-MIP were synthesized electrochemically by means of chronocoulometry or cyclic voltammetry on a portable chip having gold thin-film electrodes (TFE) system. The resulting ncovNP-MIP sensor showed a linear response to ncovNP in the lysis buffer up to 111 fM with a detection and quantification limit of 15 fM and 50 fM, respectively. The resulting ncovS1-MIP sensor showed a linear response to ncovS1 in the PBS buffer up to 200 fM with a detection and quantification limit of 15 fM and 51 fM, respectively. Both sensors remarkably sensitively recognized their specific target and discriminated interfering proteins with surprisingly excellent signal to background ratio (ncovNP-MIP discriminated S1, E2 HCV, BSA, and CD48; ncovS1-MIP discriminated NP, E2, HCV, HSA, and IgG) and signaled the presence of the specific antigen (ncovNP or ncovS1) in samples obtained from various environmental or human sources, including nasopharyngeal swab samples of COVID-19 positive patients. The presented strategy unlocks a new route for the development of rapid SARS-CoV-2 analytic tools as well as COVID-19 diagnostic tools.

### SHORT DESCRIPTION OF THE DRAWINGS

FIG. 1. Illustrates the analysis principle of portable nconNP or nconS1 sensor. Example shows COVID-19 analysis/diagnostics principle by nconNP sensor from clinical samples from nasopharyngeal swab specimens of patients. ncovNP-MIP film is integrated with TFE (thin film electrode). The sensor is incubated 15 minutes in lysis buffer supplemented with sample (here nasopharyngeal swab sample of a patient). The result is read by differential pulse voltammetry from the sensor. Here, a portable differential pulse voltammetry reader is attached to a cell phone and the result is immediately displayed to the user.

FIG. 2. Shows examples of cyclic voltammograms recorded in 1 M KCl solution containing a 4 mM redox probe K3[Fe(CN)6]/K4[Fe(CN)6] on bare Au-TFE (1), and after the subsequent fabrication steps of ncovNP-MIP: modification by 4-ATP (2) and DTSSP (3), immobilization of ncovNP (4), electrodeposition of PmPD (5) and treatment in 2-ME and acetic acid (6) (see section “Sensor fabrication” for details). Similar voltammograms were recorded for ncovS1-MIP sensor as well (in ncov-S1-MIP fabrication electrodeposition of PABA was used and the treatment was conducted in DTT and acetic acid, results not shown).

FIG. 3. Effect of charge densities applied during synthesis of PmPD on the saturated responses (I_{n.sat(MIP)}, I_{n.sat(MIP)}) of ncovNP-MIP/Au-TFE, NIP/Au-TFE and IF. The I_{n.sat(MIP)}, I_{n.sat(MW)} were derived from the corresponding adsorption isotherms (FIGS. 12A-C) and the respective IF was calculated according to Eq. 3. The adsorption isotherms were obtained upon the incubation of ncovNP-MIP/Au-TFE and NIP/Au-TFE in LB containing the different concentrations of ncovNP and fitted to Langmuir adsorption model (Eq. 2). Similar effect of charge densities applied during synthesis of PABA on the performance of the resulting ncovS1-MIP was observed (ncovS1-MIP was prepared by potential cycling and the film thickness was controlled by the number of cycles; results not shown).

FIG. 4. Optimization of time for incubation of ncovNP sensor having PmPD (poly(m-phenylenediamine) synthesized at 2 mC/cm². The responses were obtained after incubation of the sensor in LB (lysis buffer) containing 0.1 pM ncovNP. Similar optimization of time for incubation was performed by ncovS1-MIP sensor having PABA (poly 3-aminophenylboronic acid) synthesized by the optimal number of cycles (10) that equalled to about 6 mC/cm² (results not shown).

FIGS. 5A-B. (A) Calibration plot of ncovNP sensor obtained at low concentration range of ncovNP (2-111 fM) in LB. (B) Similar calibration plot in PBS was obtained for nCov S1 (27-194 fM).

FIG. 6. Selectivity test of the ncovNP sensor showing its responses against the different proteins (S1, E2 HCV, BSA, CD48 and ncovNP) spiked at concentrations (0.04, 0.07, 0.09, and 0.11 pM) in LB. Similar selectivity in PBS was observed by ncovS1 sensor at increasing concentrations 40, 60, 80, 100, 120 fM of different proteins (ncovNP, HSA, E2 HCV, IgG and ncovS1).

FIGS. 7A-B. (A) The example calibration plots of ncovNP sensors obtained against COVID-19 negative samples in UTM (universal transport medium) of four patients, 20-fold diluted with LB and spiked with 22.2, 44.4, 66.6, 111, 222, 333 fM of ncovNP. (B) The example calibration plots of ncovS1 sensor obtained against COVID-19 negative nasopharyngeal swab samples in SPS solution. Samples were diluted with PBS (1:99) and spiked with 50-400 fM of ncovS1,

FIGS. 8A-C. (A) Cross-selectivity test of ncovNP sensor showing its responses against ncovNP, S1, and mixture of ncovNP and S1 proteins in COVID-19 negative sample in UTM diluted 20-fold with LB. The concentrations of ncovNP and S1 were selected to simulate their concentration ratio in SARS-CoV-19 virus [32].

(B) Selectivity of ncovS1 sensor for ncovS1 against ncovNP in COVID-19 negative nasopharyngeal swab samples. The concentration of ncovNP was tenfold higher than the concentration of ncovS1.

(C) The ncovS1 Sensor responses to SARS-CoV-2 spike protein subunit S1 (ncovS1) and its different strains (S1 UK VOC 202012/01, S1 Brazil P1 and S1 South Africa (SA) VOC 501.V2). The dashed lines represent the LOD determined in FIG. 9B.

FIGS. 9A-B. (A) The calibration plot of ncovNP sensor obtained by averaging the data in FIG. 7A. The squares represent data points corresponding Iₙ measured by ncovNP sensor against COVID-19 positive samples in UTM 20-fold diluted with LB.

(B) The calibration plot (solid line) of ncovS1 sensor obtained by linear regression of the averaged data in FIG. 7B. P4-P8 designated gray squares represent data points corresponding to In measured by ncovS1 sensor against COVID-19 positive samples while P1, P3 black squares are those of negative samples. The error bars represent SDs of three measurements.

FIG. 10 Stability of ncovNP sensors stored for different time intervals at room temperature. The presented responses were measured against 66.6 fM ncovNP in LB by a pair of sensors right after fabrication or at various time points after 1-9 weeks storage. Similar stability was observed for ncovS1 sensors (results not shown).

FIG. 11 Shows comparison of electrochemical PmPD growth on Au-TFE and ncovNP-modified Au-TFE. Potentiostatic electrodeposition of PmPD from 10 mM mPD in PBS on bare Au and ncovNP-modified Au working electrode of TFE at 0.6 V vs Ag/AgCl/KCl.

FIGS. 12A-C shows effect of the sensing layer thickness of rebinding properties of ncovNP sensor. Adsorption isotherms of ncovNP on ncovNP-MIP- and NIP-modified sensors having the sensing layer generated by (a) 1 mC/cm², (b) 2 mC/cm², (c) 3 mC/cm².

### DESCRIPTION OF THE INVENTION

### Definitions and Abbreviations

By ncovNP it is meant SARS-CoV-2 nucleocapsid protein. The crystal structure of ncovNP is available at PDB Protein Databank as deposit number 6VYO https://www.rcsb.org/structure/6VYO. Further physical characterization of the protein is provided in the description below:

| Protein | Code in | Molecular | Molecular | Isoelectric |
|---|---|---|---|---|
| Name | PDB | weight, kDa | volume, Å³ | point |
| ncovNP | 6VYO | 45 | 85268 | 10.07 |

By ncovS1 it is meant SARS-CoV-2 S1 subunit protein. The crystal structure of ncoS1 is available at PDB Protein Databank as deposit number 6VXX. Further physical characterization of the protein is provided in the description below.

| Protein | Code in | Molecular | Molecular | Isoelectric |
|---|---|---|---|---|
| Name | PDB | weight, kDa | volume, Å³ | point |
| ncovS1 | 6VXX | 75 | 502733 | 6.04 |

By MIP it is meant molecularly imprinted polymers.

By TFE it is meant a thin film electrode consisting of a metallic surface e.g. gold as a sensing electrode deposited on an insulating support e.g. glass,

By antigen it is meant a toxin or other foreign substance which induces an i rnrnune response in the body, especially the production of antibodies.

By SARS CoV-2 antigens it is meant any biological part of the SARS CoV-2 virus that induces an immune response.

Unless defined otherwise, all technical and scientific terms used herein have the same meaning as commonly understood by one of ordinary skill in the art to which this invention belongs. Although any materials similar or equivalent to those described herein can also be used in the practice of the present invention, exemplary materials are described for illustrative purposes.

As used herein and in the appended claims, the singular form “a,” “and,” “the” include plural referents unless the context clearly dictates otherwise.

As used herein, the terms “about” and “approximately” are used interchangeably and have the meaning a person having ordinary skill in the art would readily understand.

The terms “comprises,” “comprising,” “includes,” “including,” “having” and their conjugates mean “including but not limited to.” Terms and phrases used in this application, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. Adjectives such as, e.g.. “conventional, ” “n-aditional,” “known” and terms of similar meaning should not be construed as limiting the item described to a given time period, or to an item available as of a given time. But instead, these terms should be read to encompass conventional, traditional, normal, or standard technologies that may he available, known now, or at any time in the future.

Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. The presence of broadening words and phrases such as, “one or more,” “at least;” “but not limited to,” or other like phrases in some instances shall not be read to mean that the narrower case is intended or required in instances, wherein such broadening phrases may be Asent.

The invention is now described in detail with reference to the Figures.

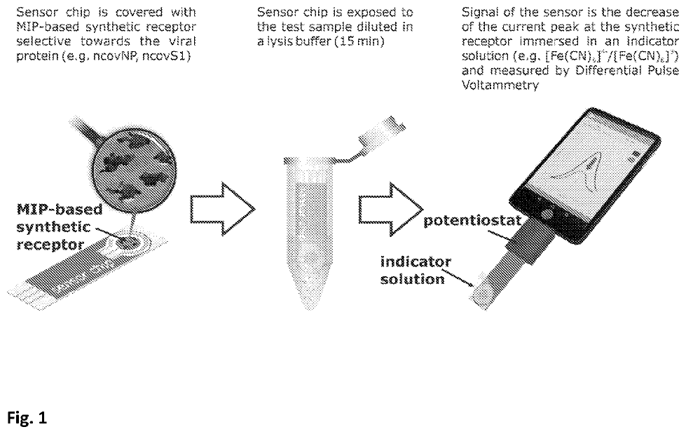

Herein, we report for the first time on the development of a MIP-based electrochemical sensor for detection of ncovNP or ncovS1 of SARS-CoV-2 (FIG. 1). The ncovNP molecularly imprinted polymer (ncovNP-MIP) or ncovS1 molecularly imprinted polymer (ncovS1-MIP) were directly generated on a portable sensing electrode, for example gold thin film electrode (TFE) surface through the electrochemical technique. After incubation in a sample solution, the prepared sensors showed remarkable capability to discriminate between the target analyte (ncovNP or ncovS1) and interfering protein such as E2 HCV, BSA, HSA, IgG and CD48 as measured by differential pulse voltammetry or square wave voltammetry in the presence of redox pair. To prove the sensor performance, the clinical diagnostic feasibility of ncovNP-MIP and ncovS1-MIP sensors was studied by analyzing nasopharyngeal swab specimens of patients.

Preferably, the sensors are prepared by modification of Au-TFE with ncovNP-MIP or ncovS1-MIP film generated from electropolymerizable monomers, selected from the group consisting of mPD, dopamine, APBA, and EDOT. Most preferably the mPD or APBA is used and the resulting polymer is poly-m-phenylenediamine (PmPD) or poly 3-aminophenylboronic acid (PABA).

The preferable thickness of the sensors determined as the thickness at which the sensor demonstrated the highest IF (imprinting effect) may be selected when the polymer film is deposited by charge density of 1-10 mC/cm², more preferably 1-7 mC/cm². Most preferably the film is deposited by charge density of 2-7 mC/cm².

The sensors according to this disclosure are highly responsive and specific for the selected antigens. The limit of detection values for the antigens is between 15 and 70 fM, more preferably 15-50 fM and most preferably 15-30fM. The values for limits of quantification of the sensors is between 50-120fM, more preferably 50-80 and most preferably about 50-51 fM. Such values are well suitable to detect antigens from COVED 19-patient samples but also from various environmental samples. Moreover, the sensors according to this disclosure are efficiently discriminating between the antigen to be measured and various interfering proteins, such as E2 HCV, CD48, HAS, IgG and BSA. Moreover, ncovNP-MIP sensor discriminates effectively S1, while ncovS1-MIP sensor discriminates effectively NP. These features ensure that the sensors, and the method provided here is accurate.

This disclosure provided a portable electrochemical sensor integrated with a molecular imprinted polymer (ncovNP-MIP and ncovS1-MIP) as a synthetic recognition element capable of selective detection of SARS-CoV-2 antigen (ncovNP or ncovS1 respectively). The synthesis parameters of sensors are configured such that the sensor has an ability to selectively rebind the antigen. The sensor has a good linearity and reproducibility in COVID-19 negative clinical samples spiked with ncovNP in the concentration range 0.22-333 fM that resulted in a LOD and LOQ values sufficient to determine the presence of ncovNP in COVID-19 positive samples from nasopharynx swab specimens, which can be expected as well as from other clinical samples or from environmental samples. For ncovS1-MIP sensor linearity response was achieved within a concentration range of 0 to 400 fM. and the LOD and LOQ were determined as 64 fM and 213 fM respectively. The developed sensor that relies on a completely different approach as compared to the currently available SARS-CoV-2 antigen tests, provides a valuable alternative as a portable diagnostic platform for the rapid screening for COVID-19 or quantitative or qualitative analysis of SARS CoV-2 antigens.

### Materials and methods

4-aminothiophenol (4-ATP), 2-mercaptoethanol (2-ME), dithiothreitol (DTT), m-phenylenediamine (mPD), bovine serum albumin (BSA), sodium dihydrogen phosphate dihydrate, disodium hydrogen phosphate, and sodium dodecyl sulfate, sodium fluoride (NaF), Immunoglobulin G (IgG), and human serum albumin (HSA) were obtained from Sigma-Aldrich. 3-aminophenylboronic acid (APBA) was obtained from Santa Cruz Biotechnology Ethanol 96% was purchased from Estonian Spirit OÜ (Estonia). SARS-Cov-2 nucleoprotein (ncovNP) and S1-subunit of SARS-Cov-2 spike protein and its different strains (S1 UK VOC 202012/01, S1 Brazil P1 and S1 South Africa VOC 501.V2) as well as CD48 protein were provided by Icosagen AS (Estonia). Hepatitis C virus (HCV) surface viral antigen (E2) was obtained from the Institute of Macromolecular Compounds of the Russian Academy of Sciences. 3,3′-dithiobis [sulfosuccinimidyl propionate] (DTSSP) and Triton X-100 were purchased from Thermo Fisher Scientific Inc., sulfuric acid, hydrogen peroxide, potassium chloride (KCl), and acetic acid were purchased from Lach-ner, S.R.O. Sodium chloride (NaCl), Tris EDTA buffer concentrate, and ethylenediaminetetraacetic acid (EDTA) were obtained from Fluka analytical. Potassium ferricyanide and ferrocyanide were purchased from Riedel-de Haen. MicruX™ thin-film electrodes (Au-TFE) were purchased from Micrux Technologies (Spain). All chemicals were of analytical grade and were used as received without any further purification. Ultrapure Milli-Q water (resistivity 18.2 MΩ cm at 25° C., EMD Millipore) was used for the preparation of all aqueous solutions. For producing the sensors and performing the analysis, named compounds could be substituted with similar chemicals from other manufacturers.

Computational Modelling Used for Selection of suitable functional monomer for ncovNP MIP

The rational selection of a suitable functional monomer for ncovNP-MIP synthesis was based on a computational modeling approach [28]. Briefly the interactions between ncovNP and candidate functional monomers mPD, dopamine (DA), and 3,4-ethylenedioxythiophen (EDOT) were modeled by using molecular docking and quantum chemical calculations. Molecular docking was performed by using Autodock 4.2.6 (from the Scripps Research Institute) on the grid-center with coordinates −13.827; 19.445; 6.256 Å to find out energetically favorable binding poses of monomers with ncovNP. The crystal structure of ncovNP (6VYO) deposited in Protein Data Bank (PDB) and monomer structures were processed within the AutoDockTools (ADT) software performing structure preparation procedures. The monomer binding poses were generated by molecular docking using Autodock4.2.6 empirical scoring function (GScore) that approximates the monomer-binding free energy and takes into account a number of non-covalent interaction parameters. Quantum chemical calculations were used to estimate the binding energies of hydrogen interactions between sterically accessible proton-accepting amino acids of ncovNP and candidate monomers and performed by using Gaussian 09. The software Rasmol (version 2.7.5.2) was used to find out the sterically accessible proton acceptor group of ncovNP (eg. polar amino acid residues). The strength of H-bond interactions between ncovNP and the monomers was calculated as a sum of association energies between a polar amino acid and the monomer as described before in [28].

The energies of prepolymerized complex structures of ncovNP with electropolymerizable monomers (mPD, dopamine, and EDOT) in aqueous solutions were determined by the computational approach. The GScore values for the best-scoring binding poses of all three monomers docked to ncovNP were almost similar ranging −25.2 and −29.5 kJ/mol. This indicated that all three monomers were able to form prepolymerization complexes with ncovNP with almost similar stability. At the same time, all the possible H-bond interactions between the respective monomers and the protein (ncovNP) in prepolymerization mixture were estimated by quantum chemical calculations. Table 1 summarizes the association energies of interaction between the polar amino acids of ncovNP and the different monomers (mPD, dopamine, and EDOT).

TABLE.1
Binding energies of ncovNP with different
monomers calculated by Gaussian 09

| Monomer | Binding energy (kJ/mol) |
|---|---|
| m-PD | 3273 |
| EDOT | 3122 |
| Dopamine | 1124 |

As it can be seen mPD forms the strongest network of H-bond interactions around ncovNP molecules as compared to the other 2 monomers. Thus, mPD monomer was selected as the optimal monomer for ncovNP-MIP synthesis.

### Sensor Fabrication

The ncovNP sensor was prepared by modification of Au-TFE with ncovNP-MIP generated from poly-m-phenylenediamine (PmPD). The protocol for synthesis of ncovNP-MIP was developed from [29]: Before modification, Au-TFE was cleaned with a cold piranha solution (H₂SO₄:H₂O₂, 3:1) for 2 minutes, then rinsed with double distilled water and dried under a nitrogen atmosphere. The working electrode of the Au-TFE was modified with 4-ATP by incubating it in 100 mM 4-ATP ethanolic solution for 30 minutes and vortex for 5 minutes in ethanol to remove the non-bound 4-ATP. 4-ATP can be substituted to any compatible amine-terminated thiol or thiocarbonyl, e.g. thiourea. The cleavable linker monolayer was generated by the covalent attachment of DTSSP (3,3′-Dithiobis(sulfosuccinimidylpropi onate)) to 4-ATP-Au-TFE via drop casting of 4 μL, (10 mM) DTSSP solution in phosphate buffered saline (PBS) for 30 minutes and washed with PBS. For ncovNP immobilization, 4 μL of PBS containing 0.55 μM of ncovNP was dropped on the cleavable linker modified electrode for 30 minutes and washed with plenty of PBS. The PBS can be substituted to any compatible buffer systems, like citrate buffer or carbonate buffer.

The synthesis of PmPD on ncovNP-modified Au-TFE was performed in the electrochemical cell (ED-AIO-Cell, Micrux Technologies, Spain) connected with the electrochemical workstation (Reference 600TM potentiostat; Gamry Instruments, USA). PmPD film was electrodeposited from PBS containing 10 mM mPD at 0.6 V vs Ag/AgCl/KCl with an optimized charge density. Molecular imprints of ncovNP in the polymer film were generated by treating the polymer film with ethanolic solution of 0.1 M 2-ME to cleave the S-S bond of DTSSP and facilitate the release of ncovNP, followed by washing with the 10% acetic acid solution. A similar procedure was also adopted for the reference film, non-imprinted polymers (NIPs), except by treating with 2-ME to save the covalent attachment of ncovNP in the polymer matrix in NIPs and to avoid formation of the molecular imprints of ncovNP in this film.

The sensor preparation steps were characterized by cyclic voltammetry (CV) in potential range of −0.2 to 0.2 at a scan rate of 100 mV/s. Cyclic voltammetry was conducted in 1 M KCl solution containing 4 mM redox probe K₃[Fe(CN)₆]/K₄[Fe(CN)₆].

Preparation ncovS1 sensor was conducted as follows:

The ncovS1 sensor was prepared by synthesizing ncovS1-MIP film directly on Au-TFE adapting the surface imprinting strategy previously developed by our group [25,26]. Prior to the modification, the Au-TFME was cleaned in ozone for 15 minutes followed by washing with ethanol, rinsing with MQ and then drying under a nitrogen atmosphere. Modification of the WE of Au-TFE was achieved by incubating it for 30 min in 100 mM 4-ATP ethanolic solution and vortexing for 5 min to remove loosely bound ATP molecules. A monolayer of cleavable linker was obtained via the covalent attachment of DTSSP to the ATP modified Au-TFE by drop casting 10 μL (10 mM) DTSSP solution in PBS for 30 min followed by washing with PBS. ncovS1 was immobilized on the DTSSP/ATP-modified Au-TFE by dropping 3 μL of PBS containing 0.33 μM of ncovS1 for 30 min and washed severally with PBS. Poly(3-aminophenylboronic acid), PAPBA was synthesized on the ncovS1-modified Au-TFE in a set-up consisting of an electrochemical cell (ED-MO-Cell, Micrux Technologies, Spain) connected with the electrochemical workstation (Reference 600TM, Gamry Instruments, USA). PAPBA was electrodeposited from a PBS solution containing 20 mM APBA and 50 mMNaF by cycling the potential between −0.2 to 0.9 V vs Ag/AgCl/KCl. Imprints of ncovS1 were generated in the polymer film by cleaving the S—S bond of DTSSP using 50 mM dithiothreitol (DTT) solution for 30 min, followed by washing for another 30 min in 10% acetic acid to remove the ncovS1. The reference, non-imprinted film (NIP), was prepared using a similar protocol but without treatment in DTT to preserve the covalently attached ncovS1 in the polymer thereby avoiding the formation of the target imprint within the matrix.

Each step of the sensor preparation was characterized by cyclic voltammetry (CV) in the potential range of −0.2 to 0.2 at a scan rate of 100 mV/s and square wave voltammetry (SWV) at a potential range of −0.2 to 0.2 V, pulse amplitude of 12.5 mV, frequency of 10 Hz, and a step potential of 5 mV in 1 M KCl solution containing 4 mM redox probe K₃[Fe(CN)₆]/K₄[Fe(CN)₆].

### Evaluation of the Sensor Performance

All the samples were prepared in a lysis buffer (LB) (pH=7.2) containing 27.5 mM of Tris-HCl, 12.5 mM of EDTA, 1.5% of Triton X-100 (V/V), and 0.1% SDS diluted with MQ water to the desired volume and stored at 4° C. The lysis buffer can be prepared by using buffering compound replacing Tris-HCl, like phosphate, acetate, citrate, carbonate, Bis-Tris, HEPES, MOPS or other systems known to skilled artisans. The EDTA can be replaced by EGTA or citrate or other chelating agents. The Triton X-100 and SDS can be replaced by other detergents, like nonidet P-40, Tween, IGEPAL or other detergent compounds. The LB did not contain a strong chaotropic agent such as guanidinium chloride in order to minimally affect the native conformations of antigen.

The rebinding of ncovNP or ncovS1 on the prepared sensors was conducted by means of DPV (differential pulse voltammetry) or SWV (square wave voltammetry). The measurements were performed in the potential range of −0.2 to 0.2 V with the pulse amplitude of 0.025 V, pulse time of 0.025 s, step potential of 0.005 V and scan rate 0.1 V/s in 1 M KCl solution containing 4 mM redox probe K₃[Fe(CN)₆]/K₄[Fe(CN)₆] at room temperature. The response (Iₙ) of the sensor was calculated as follows:

Iₙ=(I₀−I)/I₀ (1)

where I₀ and I represent the DPV or SWV anodic peak currents measured after incubation in sample solutions with and without the target analyte (ncovNP or ncovS1), respectively.

The adsorption isotherm was generated by plotting Iₙ against the respective concentration of ncovNP or ncovS1 in the measured sample solution. The response at the maximal adsorption, I_{n.sat}, was derived by fitting the isotherm to the Langmuir adsorption model (eq. 2):

Iₙ=I_{n.sat}C/(C+K_{D}) (2)

where, C is the analyte concentration, K_{D} is the dissociation constant.

The optimal thickness of the sensing layer (ncovNP-MIP or ncovS1-MIP) in the sensor was elucidated after calculation of the molecular imprinting effect or the imprinting factor (IF) for every pair of ncovNP-MIP or ncovS1-MIP- and NIP-modified sensors characterized by the same thicknesses:

IF=I_{n.sat(MIP}/I_{n.sat(NIP)} (3)

where, I_{n.sat(MIP)} and I_{n.sat(NIP)} depict I_{n.sat} responses of ncovNP-MIP or ncovS1-MIP- and NIP-modified sensors at saturation. Further, the sensors with the optimal thickness were used to determine their analytical performance in prepared and real samples solution.

The selectivity of the sensor was assessed by comparing the responses against the target (i.e. ncovNP or ncovS1) and interfering analytes (i.e., BSA, HSA, E2 HCV, IgG, and CD48) at equivalent concentrations.

Limit of Detection (LOD), and Limit of Quantitation (LOQ) of the sensors were derived from linear regression of their calibration plots obtained in LB or PBS and with for example COVID-19-negative clinical samples (see below) containing the known concentration of ncovNP or ncovS1:

LOD=3·SD/b (4)

LOD=10·SD/b (5)

where SD and b represents the standard deviation and the slope of the regression line, respectively. LOD and LOQ values can be similarly obtained for any sample of interest, including environmental samples (wastewater) or laboratory samples. As an example of clinical samples solutions of nasopharyngeal specimens in UTM (universal transport medium) from four COVID-19 negative and four COVID-19 positive patients were obtained from the SYNLAB Eesti medical laboratory (Estonia). Similarly, the clinical samples could be saliva, blood, urine, or stool samples collected by standard procedures and prepared into lysis buffer as follows. The presence or absence of the viral infection in the clinical samples was confirmed with RT-PCR method. Before the analysis, the samples were 20-fold diluted with LB by vortexing for 30 min to facilitate the viral protein release as well as to decrease the concentration of interfering species present in UTM. The samples from four COVID-19-negative patients were spiked with ncovNP at a concentration range of 22.2 -333 fM and used to construct the calibration plot and derive LOD and LOQ values.

As another example, the clinical samples obtained from SYNLAB Eesti medical laboratory (Estonia) consisted of nasopharyngeal specimens of three negative and five positive COVID-19 patients in sample preservation solution (SPS), (Jiangsu Mole Bioscience Co., Ltd). Their COVID-19 status was previously confirmed with the RT-PCR method. The samples were 100 fold diluted in PBS i.e., 1:99 (SPS:PBS). Calibration plot was plotted using the diluted negative samples spiked with an increasing concentration (0-400 fM) of ncovS1 and the LOD and LOQ values were derived. Reading DPV or SWV signals from ncovNP sensors was performed by electrochemical workstation (Reference 600TM, Gamry Instruments, USA) or a portable potentiostat (EmStat3 Blue and Sensit Smart, PalmSens BV, The Netherlands).

### Preparation and Optimization of the Sensor

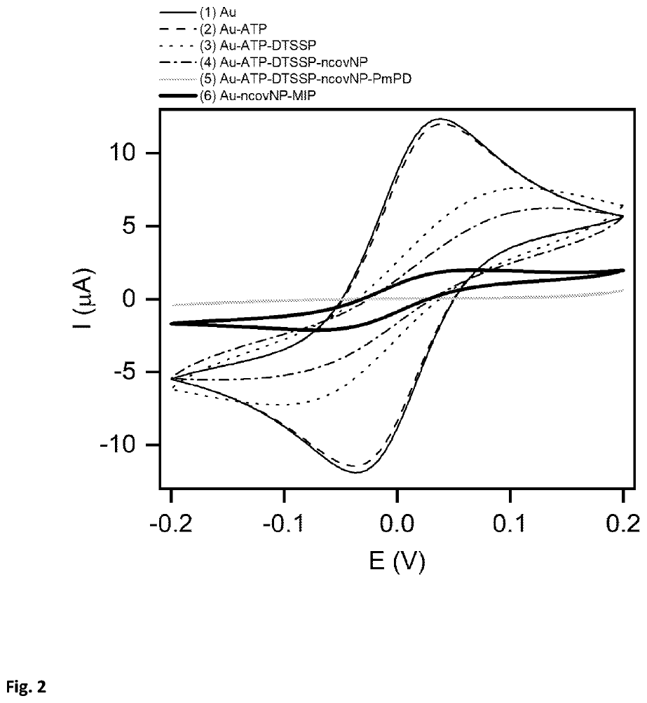

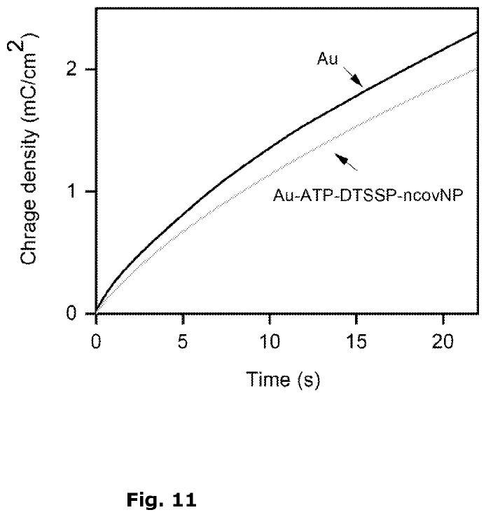

The ncovNP-MIP synthesis strategy is based on the electrochemical surface imprinting [25,29,31]. All the fabrication steps were monitored by CV (cyclic voltammogram) measurements (FIG. 2). It can be seen that while the modification of Au-TFE by 4-ATP monolayer almost did not affect anodic/cathodic current peaks of the redox couple indicating that this short thiol was too thin to effectively block electron transfer taking place on the electrode surface, on the other hand, after the subsequent attachment ncovNP via DTSSP, the redox current peaks were significantly reduced. Surprisingly, it was still possible to initiate PmPD growth at a rather low potential (0.6 V vs Ag/AgCl/KCl) comparable with that at which the similar reaction happens at a bare Au-electrode (FIG. 11). The redox peaks completely disappeared after PmPD electrodeposition indicating non-conducting polymer film formation. However, a treatment of the modified electrode in mercaptoethanol and acetic acid caused a prominent increase in the peak current indicating supposedly, elution of ncovNP from the PmPD matrix and formation of molecular cavities, which tunneled the charge transfer across the ultra-thin non-conducting PmPD layer to the electrode. Similar effects were observed during the preparation of ncovS1-MIP sensor.

Since the present imprinting approach allows the generation of macromolecular imprints situated at/or close to the polymer film surface, the deposition of polymer with an appropriate film thickness is of crucial importance in order to avoid irreversible entrapment of a protein and infeasibility of its removal during the subsequent washing out procedure. Another reason to optimize the thickness of PmPD or PABA is to provide sufficient change transfer through the imprints to the electrode for following responsive DPV sensing of protein rebinding on the resulting ncovNP-MIPs or ncov S1-MIPs.

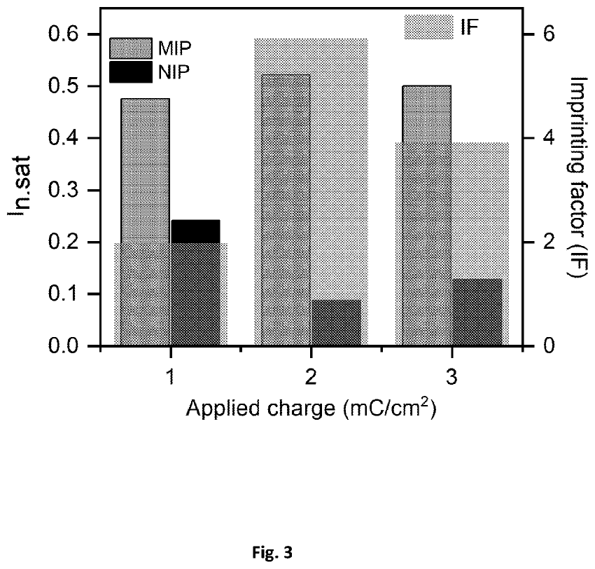

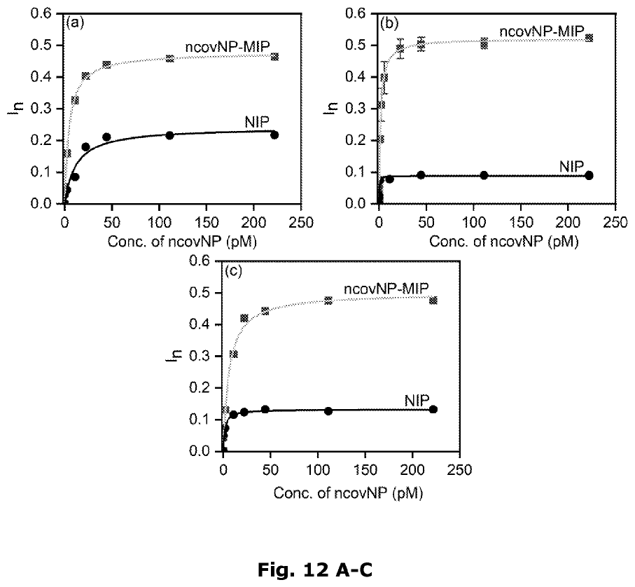

In order to determine the thickness effect of the sensing layer on the sensor, the charge densities of 1, 2, and 3 mC/cm² were imposed to generate PmPD for building ncovNP-MIP. The resulting ncovNP sensors having the different thicknesses of ncovNP-MIP were assessed in terms of their capability to rebind ncovNP from LB solutions determining IFs (Eq. 3) from analysis of the respective adsorption isotherms (FIG. 12). The appropriate thickness of ncovNP-MIP for building ncovNP sensor was determined as the thickness at which the sensor demonstrated the highest IF. As it can be seen the most pronounced imprinting effect (IF=5.8) was achieved in the case of PmPD film deposited by 2 mC/cm² (FIG. 3). Apparently, with this charge, the polymer generated at the surface of electrode confines the anchored ncovNPs, but does not hinder their successful extraction during ncovNP washing procedure. Thus, the resulting polymer is endowed with the ncovNP-selective molecular cavities capable to transduce ncovNP binding events showing the responsive inhibition of charge transfer to the modified electrode. In case of ncovS1-MIP cyclic voltammetry was used to synthesize PAPBA, charge of 6.1 mC/cm² gave an optimal polymer.

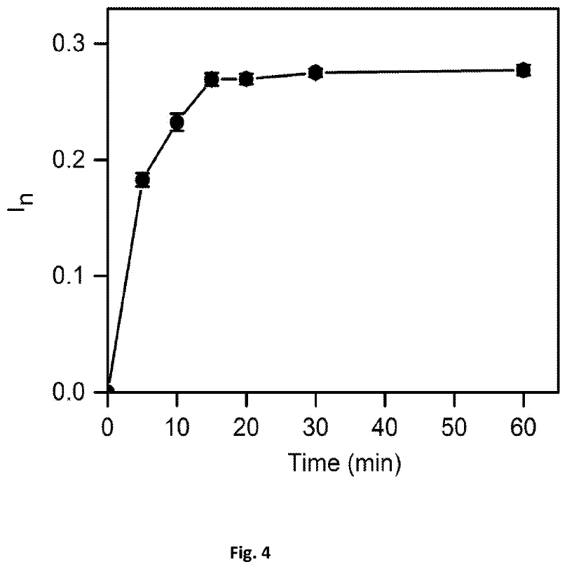

Rebinding time was another parameter that was taken into consideration. The sensor was incubated in the range of 5 and 60 minutes with LB solution containing 0.1 pM ncovNP. As it can be seen the sensor response became saturated and reached equilibrium after 15 minutes (FIG. 4). Therefore, 15 minutes was selected as the optimal time to rebind ncovNP at the surface of ncovNP sensor. Optimization of rebinding time of ncovS1 on ncovS1 was carried out by recording the responses after incubation in PBS containing 0.05 ng/mL (0.67 pM) ncovS1.

### Performance of the Sensor

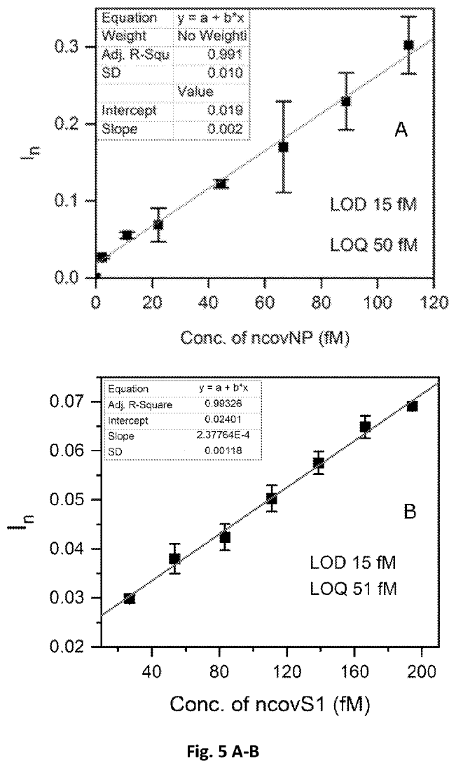

The calibration plot of ncovNP sensor against ncovNP in LB depicts the pseudo-linear increase of the sensor response with ncovNP concentration up to 111 fM (FIG. 5A). The calculated LOD and LOQ values were 15 and 50 fM (0.7-2.2 g/mL), respectively that fell well within the range of ncovNP level present in real samples of COVID-19 patients [9,32,33]. Similar calibration plot in PBS was obtained for ncov S1 (27-194 fM) (FIG. 5B).

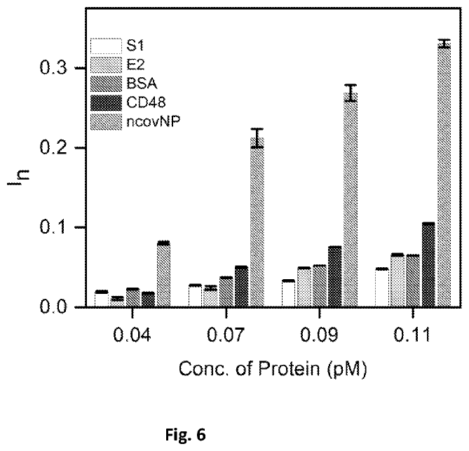

The selectivity of the ncovNP sensor against ncovNP was explored by evaluating its ability to discriminate between target and interfering proteins such as S1 (75 kDa, pI 6.0), E2 HCV (47 kDa, pI 8.2), CD48 (22 kDa, pI 9.3), and BSA (66 kDa, pI 4.7). Selectivity of ncovS1 sensor was studied against increasing concentrations (40, 60, 80, 100, and 120 fM) of different proteins (ncovNP, HSA, E2, IgG and S1) in PBS The selection of these proteins as interfering proteins was based on their size, isoelectric point, molecular weight, and possible presence in real samples. Thus, CD48 was selected due to its close isoelectric point and smaller molecular mass, E2 HCV—as a protein having molecular weight close to ncovNP, S1, being a subunit of SARS-Cov-2 spike protein, can be present in the real clinical samples along with ncovNP. As it can be seen, the response of ncovNP sensor was considerably higher against ncovNP as compared to the responses against the interfering proteins demonstrating thus the appreciable selectivity of the fabricated device towards ncovNP (FIG. 6) and promising uncompromised performance in clinical samples.

Table 2 below shows physical characteristic of the interfering proteins that were used to study the selectivity of the device.

| Protein | Code in | Molecular weight, | Molecular volume, | Isoelectric |
|---|---|---|---|---|
| Name | PDB | kDa | Å³ | point |
| CD48 | 2PTV | 22 | 16853 | 9.3 |
| BSA | 3V03 | 66 | 48160 | 4.7 |
| E2 | 4MWF | 47 | 99263 | 8.24 |
| HSA | 1E78 | 67 | 47902 | 4.7 |
| IgG | 1HZH | 152 | 223482 | 8.8 |
| ncovNP | 6VYO | 45 | 85268 | 10.07 |
| ncovS1 | 6VXX | 75 | 502733 | 6.04 |

### Sensor Performance and Stability in Clinical Samples

Performance of ncovNP sensor in clinical samples was assessed by samples prepared from nasopharyngeal swab specimens of patients as example. Samples can be originating from human source, obtained by standard clinical swab sampling techniques from mucous membranes, or from blood, urine or saliva, obtained by standard clinical sampling methods, or the samples can be obtained from environmental source, like sewage water or air to liquid sedimentation sampler, or the samples can be obtained from laboratory source, like recombinant protein expression or purification samples. For measuring the samples ncovNP or ncovS1 sensor can be used to detect NP or S1 antigens respectively.

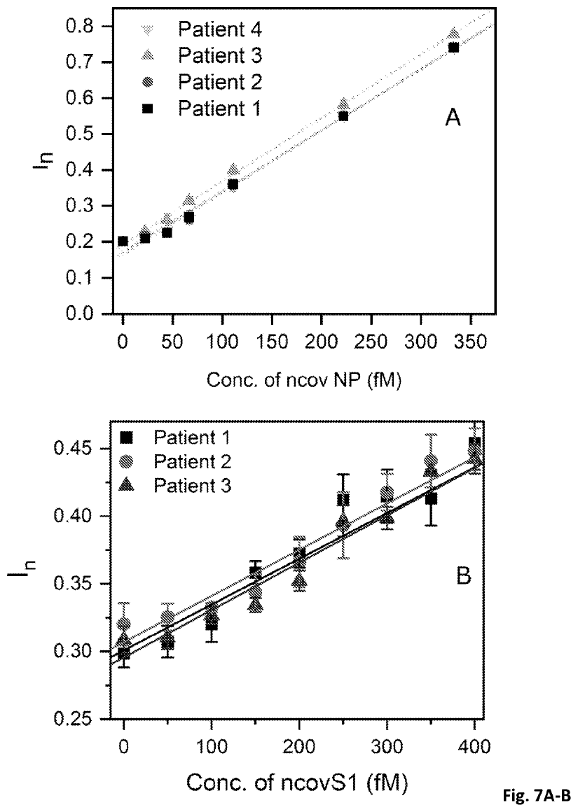

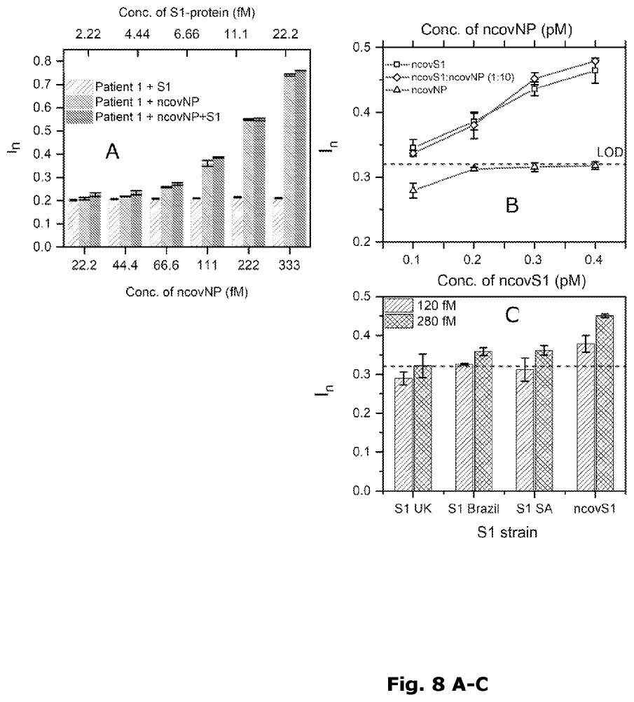

In the example, first, the sensor was calibrated in COVID-19 negative samples spiked with known concentrations of ncovNP (FIG. 7A). The sensor showed a pseudo-linear response versus ncovNP concentration in the range of 0.22-333 fM. LOD (27 fM) and LOQ (90 fM), were obtained by linearly regressing the averaged data of four COVID-19 negative patients. Thus, one can suppose that the patient' samples producing Iₙ exceeding the values of 0.22 a.u. (corresponds to LOD) can be considered as COVID-19 positive. Moreover, the sensor demonstrated appreciable selectivity to ncovNP, since its response was almost insensitive to addition of S1 in the COVID-19 negative sample, but raised immediately after ncovNP was spiked to the sample (FIG. 8A). These results promise that capability of the sensor to respond towards ncovNP will not be much disturbed in COVID-19 positive samples, where the other viral proteins can be presented. Second, the sensor was calibrated in COVID-19 negative samples spiked with known concentrations of ncovS1 in the range 50-400 fM (FIG. 7B). LOD and LOQ were determined as 64 fM and 213 fM respectively. The selectivity of the sensor was further studied in COVID-19 negative samples by spiking the sample with varying concentrations of either ncovS1, ncovNP or their mixture. The concentrations of both proteins were selected to simulate their concentration ratio, 1:10 (ncovSl:ncovNP) in SARS-CoV-2 virus [32]. As observed in FIG. 8B, the responses induced on the sensor by the increasing concentration of ncovNP are below the LOD (64 fM) indicating no recognition for ncovNP. Whereas, the sensor demonstrates remarkably increasing responses, above the LOD, to ncovS1 concentrations at tenfold lower values compared to that of ncovNP. Moreover, the responses induced by the mixture of both proteins, ncovS1:ncovNP (1:10) are comparable to that from ncovS1 thus indicating that the presence of ncovNP in the sample would not interfere, to any significant extent, with the sensor specific recognition of ncovS1 thereby enabling its accurate analysis.

To further elucidate the sensor selective recognition for the target it was tested in the samples prepared by spiking the desired amount of S1 protein of different mutated strains of the SARS-CoV-2 virus such as S1 UK VOC 202012/01, S1 Brazil P1, S1 South Africa VOC 501.V2 to COVID-19 negative samples. As seen in FIG. 8C, the sensor demonstrated the highest response for the imprinted target (ncovS1) at both concentrations. Although all strains induced a concentration-dependent sensor response, their responses, especially at the lower concentration, are analytically not significant since they fall below or are comparable to the estimated sensor's detection limit, thus highlighting the sensor's preference for ncovS1 against other strains

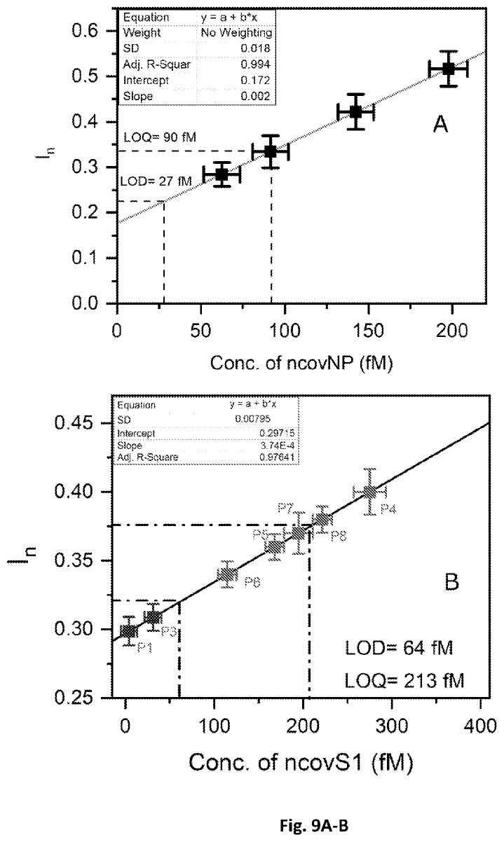

To explore the feasibility to use the sensors for COVID-19 diagnostics, they were tested against the RT-PCR confirmed COVID-19 samples. As it can be seen, four COVID-19 positive samples, obtained from the laboratory, and tested by ncovNP sensor caused the responses, In, higher than those against of the COVID-19 negative samples containing no ncovNP (y-intercept, 0.172 a.u.) or at least containing ncovNP at LOD (0.22 a.u.) (Table 3, FIGS. 9A-B).

TABLE 3
Comparison of measurements made by ncovNP-MIP sensor and
RT-PCR for COVID-19 positive nasopharynx swab specimens.

| Patient | ncovNP sensor response | ncovNP conc. |
|---|---|---|
| number (ct*) | (Iₙ)(a.u) | (fM) |
| Patient 5 (27) | 0.254 | 45 ± 11 |
| Patient 6 (27) | 0.331 | 90 ± 11 |
| Patient 7 (25) | 0.466 | 168 ± 11 |
| Patient 8 (22) | 0.544 | 214 ± 12 |

*cycle threshold value as determined by RT-PCR

It should be noted, samples of patient 6, patient 7 and patient 8, produced I. (0.331, 0.466 and 0.544 a.u.) corresponding to that caused ncovNP at concentrations higher or close to LOQ (0.33 a.u.), while Iₙ against the sample of patient 5 was higher than at LOD (0.22 a.u.). Despite the cycle threshold (Ct) values from RT-PCR cannot be directly interpreted as viral load still we observed a correlation between them and Iₙ of the sensor: the lower Ct the higher Iₙ. It is clear that the sensor is responding to the presence of viral ncovNP in COVID-19 positive samples and demonstrated thus its potential in development express tests for COVID-19.

Five COVID-19 positive samples which had been previously validated by RT-PCR were tested with the ncovS1 sensor. FIG. 9A clearly shows that all five samples effected responses that are higher (In =0.4, 0.36, 0.34, 0.37, 0.38) than LOD (In=0.32), Table 4. While samples from patients 5 and 7 produced responses close to the LOQ value, samples of patients 4 and 8 show higher responses than LOQ. The sample from patient 6 caused the lowest response, which still established a significant distinction from the LOD. Moreover, it is worthy to note that the negative samples are below the LOD. This affirms that the increased responses received from the positive patients are due to the presence of ncovS1 in the samples.

TABLE 4
ncovS1 sensor responses (Iₙ) to COVID-19 positive
samples and the associated ncovS1 concentration.

| Patient | ncovS1 sensor response | ncovS1 conc. |
|---|---|---|
| number (ct*) | (Iₙ)(a.u) | (fM) |
| Patient 4 (16) | 0.40 ± 0.02 | 280 ± 20 |
| Patient 5 (26) | 0.36 ± 0.01 | 170 ± 10 |
| Patient 6 (19) | 0.34 ± 0.01 | 120 ± 10 |
| Patient 7 (12) | 0.37 ± 0.02 | 200 ± 20 |
| Patient 8 (10) | 0.38 ± 0.01 | 220 ± 10 |

*cycle threshold value as determined by RT-PCR

### EXAMPLE: THE SENSORS HAVE AT LEAST 9 WEEKS SHELF LIFE

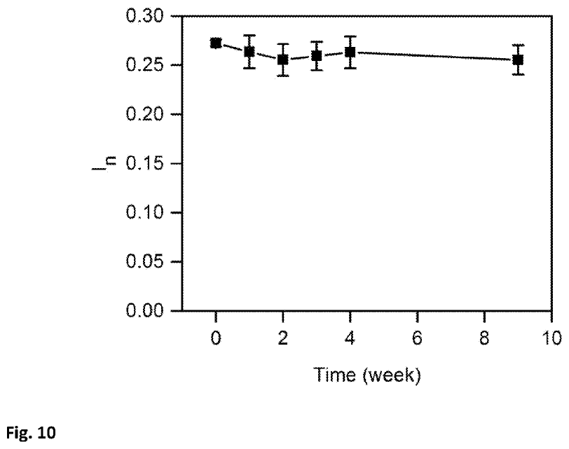

Finally, to examine the stability of the sensors, twelve (12) sensors were prepared and periodically, once a week, tested by the LB diluted COVID-19 negative samples spiked with ncovNP at concentration of 66.6 fM. The finding confirms that the response (Iₙ) of the as-prepared sensors remained the same after up to 9 weeks of storage, which reveals that ncovNP sensors have excellent long term stability (FIG. 10) i.e. it has a long shelf life.

### CONCLUSION

In this invention, we have developed for the first time a portable electrochemical sensor integrated with a molecular imprinted polymer (ncovNP-MIP and ncovS1-MIP) as a synthetic recognition element capable of selective detection of SARS-CoV-2 antigen (ncovNP or S1 respectively). The synthesis parameters of sensors were optimized and the ability of the prepared sensor to selectively rebind the antigen was demonstrated. As an example, the sensor demonstrated a good linearity and reproducibility in COVID-19 negative clinical samples spiked with ncovNP in the concentration range 0.22-333 fM that resulted in a LOD and LOQ values sufficient to determine the presence of ncovNP in COVID-19 positive samples of nasopharynx swab specimens. In addition, the sensor demonstrated a good linearity and reproducibility in COVID-19 negative clinical samples spiked with ncovS1 in the concentration range 50-400 fM that resulted in a LOD and LOQ values sufficient to determine the presence of ncovNP in COVID-19 positive samples of nasopharynx swab specimens. It is foreseen that the sensor could be used in detecting COVID-19 positive samples of other complex biological specimens.

The developed sensor that relies on a completely different approach as compared to the currently available SARS-CoV-2 antigen tests, could represent a valuable alternative as a portable diagnostic platform for the rapid screening for COVID-19.

It will be readily understood by one of ordinary skill in the relevant art that the present invention has broad utility and application. Although the present invention has been described and illustrated herein with referred to certain embodiments, it will be apparent to those of ordinary skill in the art that other embodiments may perform similar functions and/or achieve like results, and that the described embodiments are for illustrative purposes only. Thus, it should be understood that various features and aspects of the disclosed embodiments can be combined with, or substituted for one another in order to form varying modes of the disclosed invention. Many different embodiments such as, variations, adaptations, modifications, and equivalent arrangements are implicitly and explicitly disclosed by the embodiments described herein, and thus fall within the scope and spirit of the present invention.

Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method, product or use of the invention, and vice versa.

Further, the discussed prior art is not an admission by Applicant and should not be construed that the current invention does not antecede and is not patentable over the discussed prior art, but has merely been presented to better define the knowledge in the field to a skilled artisan and to the reader in general.

### ACKNOWLEDGEMENT

This research was supported by Estonian Research Council grants: COVSG34 and PRG307.

### REFERENCES

[1] P. Zhou, X.-L. Yang, X.-G. Wang, B. Hu, L. Zhang, W. Zhang, H.-R. Si, Y. Zhu, B. Li, C.-L. Huang, A pneumonia outbreak associated with a new coronavirus of probable bat origin, Nature. 579 (2020) 270-273.
[2] World Health Organization, Coronavirus disease 2019 (COVID-19): situation report, 52, (2020). https://www.who.int/docs/default- source/coronavirus e/situati on-reports/20200312-sitrep-52-covid-19.pdf? sfvrsn=e2bfc9c0_4 (accessed Oct. 30, 2020).
[3] K. Mao, H. Zhang, Z. Yang, Can a paper-based device trace COVID-19 sources with wastewater-based epidemiology?, (2020).
[4] D. Wrapp, N. Wang, K. S. Corbett, J. A. Goldsmith, C.-L. Hsieh, O. Abiona, B. S. Graham, J. S. McLellan, Cryo-EM structure of the 2019-nCoV spike in the prefusion conformation, Science. 367 (2020) 1260-1263.
[5] W. Liu, L. Liu, G. Kou, Y. Zheng, Y. Ding, W. Ni, Q. Wang, L. Tan, W. Wu, S. Tang, Evaluation of Nucleocapsid and Spike Protein-based ELISAs for detecting antibodies against SARS-CoV-2, J. Clin. Microbiol. (2020).
[6] C. Chang, S.-C. Sue, T. Yu, C.-M. Hsieh, C.-K. Tsai, Y.-C. Chiang, S. Lee, H. Hsiao, W.-J. Wu, W.-L. Chang, Modular organization of SARS coronavirus nucleocapsid protein, J. Biomed. Sci. 13 (2006) 59-72.
[7] K. R. Hurst, C. A. Koetzner, P. S. Masters, Identification of in vivo-interacting domains of the murine coronavirus nucleocapsid protein, J. Virol. 83 (2009) 7221-7234.
[8] L. Cui, H. Wang, Y. Ji, J. Yang, S. Xu, X. Huang, Z. Wang, L. Qin, P. Tien, X. Zhou, The nucleocapsid protein of coronaviruses acts as a viral suppressor of RNA silencing in mammalian cells, J. Virol. 89 (2015) 9029-9043.
[9] T. Li, L. Wang, H. Wang, X. Li, S. Zhang, Y. Xu, W. Wei, Serum SARS-COV-2 Nucleocapsid Protein: A Sensitivity and Specificity Early Diagnostic Marker for SARS-COV-2 Infection, MedRxiv. (2020).
[10] C.-C. Lai, C.-Y. Wang, W.-C. Ko, P.-R. Hsueh, In vitro diagnostics of coronavirus disease 2019: technologies and application, J. Microbiol. Immunol. Infect. (2020).
[11] S. Chen, D. Lu, M. Zhang, J. Che, Z. Yin, S. Zhang, W. Zhang, X. Bo, Y. Ding, S. Wang, Double-antigen sandwich ELISA for detection of antibodies to SARS-associated coronavirus in human serum, Eur. J. Clin. Microbiol. Infect. Dis. 24 (2005) 549-553.
[12] Y. Liu, Y. Liu, B. Diao, F. Ren, Y. Wang, J. Ding, Q. Huang, Diagnostic Indexes of a Rapid IgG/IgM Combined Antibody Test for SARS-CoV-2, MedRxiv. (2020).
[13] Find.Test directory, FIND. (n.d.). https://www.finddx.org/test-directory/(accessed Apr. 13, 2021).
[14] L. Porte, P. Legarraga, V. Vollrath, X. Aguilera, J. M. Munita, R. Araos, G. Pizarro, P. Vial, M. Iruretagoyena, S. Dittrich, Evaluation of novel antigen-based rapid detection test for the diagnosis of SARS-CoV-2 in respiratory samples, Int. J. Infect. Dis. (2020).
[15] G. Seo, G. Lee, M. J. Kim, S.-H. Baek, M. Choi, K. B. Ku, C.-S. Lee, S. Jun, D. Park, H. G. Kim, Rapid detection of COVID-19 causative virus (SARS-CoV-2) in human nasopharyngeal swab specimens using field-effect transistor-based biosensor, ACS Nano. 14 (2020) 5135-5142.
[16] Sofia SARS Antigen FIA, (n.d.). https://www.fda.gov/media/137885/download (accessed Nov. 2, 2020).
[17] K. Haupt, K. Mosbach, Molecularly imprinted polymers and their use in biomimetic sensors, Chem. Rev. 100 (2000) 2495-2504.
[18] W. Zhao, B. Li, S. Xu, X. Huang, J. Luo, Y. Zhu, X. Liu, Electrochemical protein recognition based on macromolecular self-assembly of molecularly imprinted polymer: a new strategy to mimic antibody for label-free biosensing, J. Mater. Chem. B. 7 (2019) 2311-2319.
[19] S. Viswanathan, C. Rani, S. Ribeiro, C. Delerue-Matos, Molecular imprinted nanoelectrodes for ultra sensitive detection of ovarian cancer marker, Biosens. Bioelectron. 33 (2012) 179-183.
[20] P. Jolly, V. Tamboli, R. L. Harniman, P. Estrela, C. J. Allender, J. L. Bowen, Aptamer-MIP hybrid receptor for highly sensitive electrochemical detection of prostate specific antigen, Biosens. Bioelectron. 75 (2016) 188-195.
[21] Y. Wang, Z. Zhang, V. Jain, J. Yi, S. Mueller, J. Sokolov, Z. Liu, K. Levon, B. Rigas, M. H. Rafailovich, Potentiometric sensors based on surface molecular imprinting: Detection of cancer biomarkers and viruses, Sens. Actuators B Chem. 146 (2010) 381-387.
[22] V. V. Shumyantseva, T. V. Bulko, L. V. Sigolaeva, A. V. Kuzikov, A. I. Archakov, Electrosynthesis and binding properties of molecularly imprinted poly-o-phenylenediamine for selective recognition and direct electrochemical detection of myoglobin, Biosens. Bioelectron. 86 (2016) 330-336.
[23] B. V. Silva, B. A. Rodriguez, G. F. Sales, T. S. Maria Del Pilar, R. F. Dutra, An ultrasensitive human cardiac troponin T graphene screen-printed electrode based on electropolymerized-molecularly imprinted conducting polymer, Biosens. Bioelectron. 77 (2016) 978-985.
[24] A. Kidakova, J. Reut, R. Boroznjak, A. Opik, V. Syritski, Advanced sensing materials based on molecularly imprinted polymers towards developing point-of-care diagnostics devices., Proc. Est. Acad. Sci. 68 (2019).
[25] A. Kidakova, R. Boroznjak, J. Reut, A. Opik, M. Saarma, V. Syritski, Molecularly imprinted polymer-based SAW sensor for label-free detection of cerebral dopamine neurotrophic factor protein, Sens. Actuators B Chem. 308 (2020) 127708.
[26] C.-H. Lu, Y. Zhang, S.-F. Tang, Z.-B. Fang, H.-H. Yang, X. Chen, G.-N. Chen, Sensing HIV related protein using epitope imprinted hydrophilic polymer coated quartz crystal microbalance, Biosens. Bioelectron. 31 (2012) 439-444.
[27] D.-F. Tai, C.-Y. Lin, T.-Z. Wu, L.-K. Chen, Recognition of dengue virus protein using epitope-mediated molecularly imprinted film, Anal. Chem. 77 (2005) 5140-5143.
[28] R. Boroznjak, J. Reut, A. Tretjakov, A. Lomaka, A. Opik, V. Syritski, A computational approach to study functional monomer-protein molecular interactions to optimize protein molecular imprinting, J. Mol. Recognit. 30 (2017) e2635.
[29] A. Tretjakov, V. Syritski, J. Reut, R. Boroznjak, A. Opik, Molecularly imprinted polymer film interfaced with Surface Acoustic Wave technology as a sensing platform for label-free protein detection, Anal. Chim. Acta. 902 (2016) 182-188.
[30] M. Scallan, C. Dempsey, J. Macsharry, Validation of a Lysis Buffer Containing 4 M Guanidinium Thiocyanate (GITC), Triton X-100 Extr. SARS-CoV-2 RNA COVID-19 Test. Comp. Formul. Lysis Buffers Contain. 4 (n.d.) 2020-04.
[31] A. Tretjakov, V. Syritski, J. Reut, R. Boroznjak, O. Volobujeva, A. Opik, Surface molecularly imprinted polydopamine films for recognition of immunoglobulin G, Microchim. Acta. 180 (2013) 1433-1442.
[32] Y. M. Bar-On, A. Flamholz, R. Phillips, R. Milo, Science Forum: SARS-CoV-2 (COVID-19) by the numbers, Elife. 9 (2020) e57309.
[33] Y. Pan, D. Zhang, P. Yang, L. L. Poon, Q. Wang, Viral load of SARS-CoV-2 in clinical samples, Lancet Infect. Dis. 20 (2020) 411-412.
[34] M. S. Han, J.-H. Byun, Y. Cho, J. H. Rim, RT-PCR for SARS-CoV-2: quantitative versus qualitative, Lancet Infect. Dis. (2020) S1473309920304242. https://doi.org/10.1016/S1473-3099(20)30424-2.
[35] Y. Cai, J. Zhang, T. Xiao, H. Peng, S. M. Sterling, R. M. Walsh Jr, S. Rawson, S. Rits-Volloch, B. Chen, Distinct conformational states of SARS-CoV-2 spike protein, Science 369:6511 (2020) 1586-1592.
[36] L. Dai & G. F. Gao, Viral targets for vaccines against COVID-19, Nature Reviews Immunology 21 (2021) 73-82.
[37] J. Peccia, A. Zulli, D. E. Brackney, N. D. Grubaugh, E. H. Kaplan, A. Casanovas-Massana, A. I. Ko, A. A. Malik, D. Wang, M. Wang, J. L. Warren, D. M. Weinberger, W. Arnold & S. B. Omer, Measurement of SARS-CoV-2 RNA in wastewater tracks community infection dynamics, Nature Biotechnology 38 (2020) 1164-1167.

1. A detection assembly for detecting at least one SARS-CoV-2 antigen from a sample, the assembly comprising portable electrochemical sensor integrated with a SARS-CoV-2 antigen-molecular imprinted polymer (MIP), wherein the SANS-CoV-2 antigen-MIP is configured to act as a synthetic recognition element selectively detecting and binding the at least one SARS-CoV-2 antigen; and a reading device capable of measuring presence of absence of the antigen.

2. The detection assembly of claim 1, wherein the at least one SARS-CoV-2 antigen is SARS CoV-2 nucleoprotein ncovNP car SARS CoV-2 S1-subunit protein ncovS1.

3. The detection assembly of claim 1, wherein the sample is from an environmental source, such as sewage water, or air to liquid sedimentation sampler; from a human source, such as a swabbing sample from mucous membranes, or blood, urine or saliva sample; or from a laboratory source, such as a recombinant protein expression or purification sample.

4. The detection assembly of claim 1, wherein the detection assembly has a detection limit in a range of 15 to 70 fM, more preferably' 15 to 50 fM, and most preferably 15-30fM.

5. The detection assembly of claim 1, wherein the detection assembly has a quantification limit of 50 to 220 fM, more preferabl 50 to 80 fM and most preferably about 5-51 fM.

6. The detection assembly of claim 1, wherein the assembly has a shelf e more than weeks, preferably more than 7 weeks, and most preferably at least 9 weeks.

7. The detection assembly of claim 1, wherein the detection assembly is configured to discriminate at least E2 (E2 envelope protein of Hepatitis C virus), HCV (Hepatitis C virus antigens), BSA (bovine serum albumin), HSA (human serum albumin), IgG (immunoglobulin (G) and CD48 (Cluster of Differentiation 48) proteins.

8. The detection assembly of claim 1, wherein a polymeric layer coating of the electrochemical sensor is formed from electropolymerizable monomers selected from the group consisting of mPD, 3-aminophenylboronic acid (APBA), dopamine and EDOT; most preferably mPD or APBA, and the polymeric layer coating is deposited by 1-10 mC/cm²; more preferably 1-7 mC/cm², and most preferably by 2-7 mC/cm².

9. The detection assembly of claim 1, wherein the detection assembly comprises a potentiostat reader.

10. The detection assembly of claim 9, wherein the potentiostat eader is connectable to a cellular phone.

11. A method to detect presence or absence or quantitative concentration of at least one SARS-CoV-2 antigen in a sample, wherein the method comprises: providing a sensor comprising SARS-Coe-2 antigen molecular imprinted polymer (MIP) integrated with a sensing electrode, such as a thin film electrode (TFE); bringing the sensor in contact with the sample;
and detecting the presence or absence of the antigen by differential pulse voltammetry (DPV) or square voltammetry (SWV), wherein presence of the antigen is recorded when limit of detection (LOD) of the sensor is exceeded.

12. The method of claim 11, wherein bringing the sensor in contact with the sample is obtained by incubating the sensor in a buffer, optionally containing a detergent, and comprising the sample, preferably less than 60 minutes, more preferably less than 30 minutes, even more preferably between 20 and 30 minutes, and most preferably 15-20 minutes.

13. The method of claim 11, wherein the antigen is SARS CoV-2 nucleoprotein ncovNP or SARS CoV-2 S1-subunit protein ncovS1.

14. The method of claim 11, wherein the sample is obtained from an environmental source, such as sewage water, or air to liquid sedimentation sampler; from a human source, such as a swabbing sample from mucous membranes, or blood, urine or saliva sample; or from a laboratory source, such as a recombinant protein expression or purification sample.

15. A method for making an electrochemical sensor for detection SARS-CoV-2 antigen, especially ncovNP or ncovS1 from samples, the method comprising the steps of:
forming a cleavable linking layer on a metallic surface of sensing electrode surface deposited on an insulating support;
immobilizing ncovNP and/or ncovS1 molecules on the cleavable linking layer on the electrode surface;
polymerizing a polymer on the antigen -immobilized electrode surface thereby forming a polymeric layer coating on the electrode surface with entrapped antigen molecules;
and cleaving off the cleavable linking layer thereby removing the antigen molecules from the polymeric layer and obtaining antigen sensor configured to capture antigens similar to those removed by cleaving off the linking layer.

16. The method of claim 15, wherein the polymer is polymerized from electropolymerizable monomers selected from the group consisting of m-phenylenediamine (mPD), 3-aminophenylboronic acid (APBA), dopamine and 3,4-ethylenedioxythiophene (EDOT), most preferably mPD or APBA.
